(19) United States
(12) Patent Application Publication
(10) Pub. No.: US 2026/0108296 A1
(43) Pub. Date: Apr. 23, 2026

(54) SYSTEM AND METHOD FOR IDENTIFYING CRITICAL ISTHMUS

(71) Applicant: St. Jude Medical, Cardiology Division, Inc., St. Paul, MN (US)

(72) Inventors: Zeren Su Yildiz, St. Paul, MN (US); Shuaidong Zhao, Inver Grove Heights, MN (US); Hana Y. Boudlali, Blaine, MN (US); Hai Huang, Woodbury, MN (US)

(21) Appl. No.: 19/327,637

(22) Filed: Sep. 12, 2025

Related U.S. Application Data
(60) Provisional application No. 63/708,567, filed on Oct. 17, 2024.

(51) Int. Cl. A61B 18/14; A61B 5/339; A61B 5/363; A61B 5/367; A61B 18/00

(52) U.S. Cl. CPC A61B 18/1492; A61B 5/339; A61B 5/363; A61B 5/367; A61B 2018/00351; A61B 2018/00577; A61B 2018/00839; A61B 2018/00892

(57) ABSTRACT

Critical isthmus for treatment of tachycardia via application of ablation therapy may be automatically identified by an electroanatomical mapping system from voltage and timing maps of a portion of a patient's heart. The system identifies one or more low-voltage regions from the voltage map and one or more deceleration zones from the timing map. Based on the identified low-voltage regions and deceleration zones, the system can generate an output including an identification of one or more critical isthmus locations through application of a classifier trained with data from a plurality of subjects that received ablation therapy for treatment of tachycardia. The system can also output a tachycardia termination confidence value for each identified critical isthmus location. The classifier may employ a convolutional neural network algorithm.

### CROSS-REFERENCE TO RELATED APPLICATIONS

This application claims the benefit of U.S. provisional application No. 63/708,567, filed 17 Oct. 2024, which is hereby incorporated by reference as though fully set forth herein.

### FIELD

The present disclosure relates generally to electrophysiology procedures. In particular, the present disclosure relates to systems, apparatuses, and methods for identifying critical isthmus for termination of tachycardia via ablation therapy.

### BACKGROUND

Electrophysiological mapping, and more particularly electrocardiographic mapping, is a part of numerous cardiac diagnostic and therapeutic procedures. Such mapping includes measuring the electrical activity of the heart over time using surface leads and/or intracardiac measurement electrodes.

In conjunction with electrophysiological mapping for diagnosis, ablation therapy may be used to treat various conditions afflicting the human anatomy. One condition in which ablation therapy may be used is the treatment of tachycardia.

For instance, it is known that critical isthmus sites are desirable targets for the application of ablation therapy for tachycardia termination. Thus, electrophysiological mapping may be used to identify critical isthmus sites, and ablation therapy may be applied at the identified critical isthmus sites to terminate tachycardia.

### BRIEF SUMMARY

The instant disclosure provides a computer-implemented method of identifying critical isthmus for treatment of tachycardia via application of ablation therapy. The method includes: receiving a voltage map of a portion of a patient's heart and a timing map of the portion of the patient's heart at an electroanatomical mapping system; identifying, using the electroanatomical mapping system, one or more low-voltage regions in the portion of the patient's heart from the voltage map; identifying, using the electroanatomical mapping system, one or more deceleration zones in the portion of the patient's heart from the timing map; and generating an output, based on the identified one or more low-voltage regions in the portion of the patient's heart and the identified one or more deceleration zones in the portion of the patient's heart, using a critical isthmus processing unit including a classifier trained with data from a plurality of subjects that received ablation therapy for treatment of tachycardia, wherein the output includes an identification of one or more critical isthmus locations in the portion of the patient's heart.

The output may further include a tachycardia termination confidence value for each of the one or more identified critical isthmus locations.

The step of identifying, using the electroanatomical mapping system, one or more low-voltage regions in the portion of the patient's heart from the voltage map can include the electroanatomical mapping system identifying one or more regions having a peak-to-peak voltage within a preset range as the one or more low-voltage regions. It is contemplated that the preset range may be between 0.5 mV and 1.5 mV and that the preset range may be set or adjusted via a user input received at the electroanatomical mapping system.

The step of identifying, using the electroanatomical mapping system, one or more deceleration zones in the portion of the patient's heart from the timing map can include the electroanatomical mapping system identifying one or more regions of a preset size including a greatest number of isochronal groups as the one or more deceleration zones. It is further contemplated that the timing map can include a preset number of isochronal groups and each of the one or more deceleration zones can include at least half of the preset number of isochronal groups.

The preset size may be a 1 cm radius sphere. Alternatively, the preset size may be set or adjusted via a user input received at the electroanatomical mapping system.

The critical isthmus processing unit can include a convolutional neural network algorithm.

Also disclosed herein is an electroanatomical mapping system for identifying critical isthmus for treatment of tachycardia via application of ablation therapy. The electroanatomical mapping system includes: a critical isthmus processing unit including a classifier trained with data from a plurality of subjects that received ablation therapy for treatment of tachycardia. The critical isthmus processing unit is configured to: receive a voltage map of a portion of a patient's heart and a timing map of the portion of the patient's heart; identify one or more low-voltage regions in the portion of the patient's heart from the voltage map; identify one or more deceleration zones in the portion of the patient's heart from the timing map; and generate an output, based on the identified one or more low volage regions in the portion of the patient's heart and the identified one or more deceleration zones in the portion of the patient's heart, using the classifier, wherein the output includes an identification of one or more critical isthmus locations in the portion of the patient's heart.

The output can further include a tachycardia termination confidence value for each of the one or more identified critical isthmus locations.

Each low-volage region of the one or more low-voltage regions includes a peak-to-peak voltage within a preset range, such as between 0.5 mV and 1.5 mV.

Each deceleration zone of the one or more deceleration zones includes, within a region of the portion of the patient's heart having a preset size, a greatest number of isochronal groups. Each deceleration zone of the one or more deceleration zones may also include, within the region of the patient's heart having the preset size, at least half of a preset total number of isochronal groups. The region of the portion of the patient's heart having the preset size may be a 1 cm radius spherical region of the portion of the patient's heart.

The critical isthmus processing unit can include a convolutional neural network algorithm.

The instant disclosure also provides a method of using an electroanatomical mapping system to identify critical isthmus for treatment of tachycardia via application of ablation therapy. The method includes: providing the electroanatomical mapping system with a critical isthmus processing unit, wherein the critical isthmus processing unit includes a classifier trained with data from a plurality of subjects that received ablation therapy for treatment of tachycardia. The critical isthmus processing unit is further programmed to execute a series of steps including: receiving a voltage map of a portion of a patient's heart and a timing map of the portion of the patient's heart; classifying the portion of the patient's heart into one or more scar regions, one or more healthy regions, and one or more low-volage regions using the electroanatomical mapping system using the voltage map of the portion of the patient's heart; identifying one or more deceleration zones in the portion of the patient's heart using the timing map of the portion of the patient's heart; and generating an output using the classifier, wherein the output includes an identification of one or more critical isthmus locations in the portion of the patient's heart based on the identified one or more low volage regions in the portion of the patient's heart and the identified one or more deceleration zones in the portion of the patient's heart.

The output can further include a tachycardia termination confidence value for each of the one or more identified critical isthmus locations.

The instant disclosure also provides a computer-implemented method of identifying critical isthmus for treatment of tachycardia via application of ablation therapy. The method comprises: receiving a voltage map of a portion of a patient's heart and a timing map of the portion of the patient's heart at an electroanatomical mapping system; identifying, using the electroanatomical mapping system, one or more low-voltage regions in the portion of the patient's heart from the voltage map; identifying, using the electroanatomical mapping system, one or more deceleration zones in the portion of the patient's heart from the timing map; and generating an output that comprises an identification of one or more critical isthmus locations in the portion of the patient's heart using a trained classifier and based on the identified one or more low-voltage regions in the portion of the patient's heart and the identified one or more deceleration zones in the portion of the patient's heart.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIG. 2 is a flowchart of representative steps that can be carried out according to aspects of the instant disclosure. 6

FIG. 3A illustrates a voltage map, and more particularly a peak-to-peak voltage map, as may be used in accordance with the teachings herein.

FIG. 3B illustrates a timing map, and more particularly an isochronal late activation map (ILAM), as may be used in accordance with the teachings herein.

FIG. 4A is a close-up view of box 400A in FIG. 3A.

FIG. 4B is a series of close-up views of box 400B in FIG. 3B.

FIG. 5 illustrates how deceleration of a cardiac activation wavefront can be visualized in a timing map.

FIG. 6 illustrates an annotation of a critical isthmus location on the voltage and timing maps of FIGS. 3A and 3B.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides systems, apparatuses, and methods for identifying critical isthmus locations. For purposes of illustration, aspects of the disclosure will be described with reference to the use of a suitably-programmed electroanatomical mapping system, such as the EnSite Precision™ cardiac mapping system (Abbott Laboratories, Abbott Park, IL), to identify critical isthmus locations for the termination of tachycardia via application of ablation therapy. Those of ordinary skill in the art will understand, however, how to apply the teachings herein to good advantage in other contexts and/or with respect to other devices and cardiac rhythm disorders.

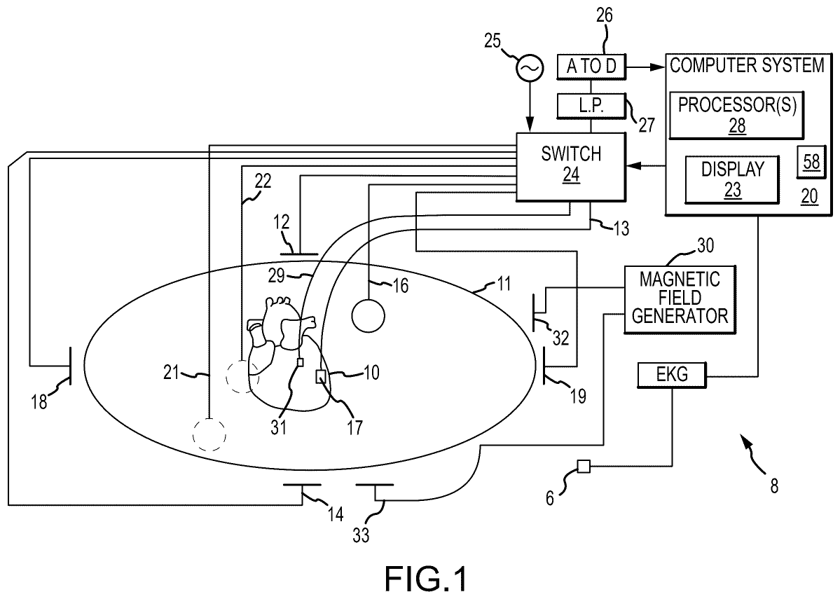

FIG. 1 shows a schematic diagram of an exemplary electroanatomical mapping system 8 for conducting cardiac electrophysiology studies by navigating a cardiac catheter and measuring electrical activity occurring in a heart 10 of a patient 11 and mapping the electrical activity and/or information related to or representative of the electrical activity so measured. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example to create a diagnostic data map of the patient's heart 10.

As one of ordinary skill in the art will recognize, system 8 determines the location, and in some aspects the orientation, of objects, typically within a three-dimensional space, and expresses those locations as position information determined relative to at least one reference. This is referred to herein as “localization.”

For simplicity of illustration, the patient 11 is depicted schematically as an oval. In the embodiment shown in FIG. 1, surface electrodes (e.g., patch electrodes) 12, 14, 16, 18, 19, and 22 are shown applied to a surface of the patient 11, pairwise defining three generally orthogonal axes, referred to herein as an x-axis (12, 14), a y-axis (18, 19), and a z-axis (16, 22). In other embodiments, the surface electrodes could be positioned in other arrangements, for example multiple electrodes on a particular body surface. As a further alternative, the electrodes do not need to be on the body surface, but could be positioned internally to the body or on an external frame.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 16, 22 are applied to the patient along a second axis generally orthogonal to the x-axis, along the sternum and spine of the patient in the thorax region, and may be referred to as the Chest and Back electrodes. The z-axis electrodes 18, 19 are applied along a third axis generally orthogonal to both the x-axis and the y-axis, such as along the inner thigh and neck regions of the patient, and may be referred to as the Left Leg and Neck electrodes. The heart 10 lies between these pairs of surface electrodes 12/14, 16/22, and 18/19.

Each surface electrode can measure multiple signals. For example, in embodiments of the disclosure, each surface electrode can measure three resistance (impedance) signals and three reactance signals. These signals can, in turn, be grouped into three resistance/reactance signal pairs. One resistance/reactance signal pair can reflect driven values, while the other two resistance/reactance signal pairs can reflect non-driven values (e.g., measurements of the electric field generated by other driven pairs in a manner similar to that described below for electrodes 17).

An additional surface reference electrode (e.g., a “belly patch”) 21 provides a reference and/or ground electrode for the system 8. The belly patch electrode 21 may be an alternative to a fixed intra-cardiac electrode 31, described in further detail below. In alternative embodiments where system 8 is capable of magnetic field-based localization instead of or in addition to impedance-based localization, the surface electrode 21 can alternatively or additionally include a magnetic patient reference sensor-anterior (“PRS-A”) positioned on the patient's chest.

It should be appreciated that patient 11 may also have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. In other embodiments, system 8 may utilize a single catheter that includes multiple splines, each of which in turn includes multiple electrodes, such as the Advisor™ HD Grid Mapping Catheter, Sensor Enabled™ (Abbott Laboratories; Abbott Park, Illinois). These embodiments are merely exemplary, however, and any number of electrodes and/or catheters may be used.

As the ordinarily-skilled artisan will appreciate, electrodes 17 carried in the distal portion of catheter 13 can be used to measure unipolar electrograms. Unipolar electrograms have the advantage of being orientation-independent (that is, a given electrode 17 will measure substantially the same unipolar electrogram regardless of the orientation of catheter 13 relative to the cardiac surface). On the other hand, unipolar electrograms often include not only the component of interest (e.g., a near-field cardiac activation component), but also various far-field noise components. These far-field noise components include, but are not limited to, far-field cardiac activation components, powerline noise components, patient respiration components, patient motion components, and cardiac motion components. Unipolar electrograms also ignore local directional information in the form of the ion currents that produce electrograms.

It may also be desirable to measure bipolar electrograms, which are less susceptible to far-field noise than unipolar electrograms and incorporate directional information along their axes. As those of ordinary skill in the art will recognize, any two neighboring electrodes 17 on catheter 13 define a bipole. Any bipole can, in turn, be used to generate a bipolar electrogram according to techniques that will be familiar to those of ordinary skill in the art. Those of ordinary skill in the art will recognize, however, that bipolar electrograms are orientation-dependent (that is, they will change as the orientation of catheter 13 relative to the cardiac surface changes).

There are also extant techniques that allow bipolar electrograms to be combined to generate electrograms for any orientation of catheter 13 relative to the cardiac surface without physically changing the orientation of catheter 13. These orientation-independent techniques are often referred to as “omnipolar” techniques. In turn, the computed electrogram signals that result from the application of such techniques can be referred to as “omnipolar electrograms” or “virtual bipolar electrograms.” These omnipolar electrograms can be thought of as the bipolar electrogram that would be seen by an “omnipole” or “virtual bipole” having its “omnipole orientation” or “virtual bipole orientation” at a particular angle relative to the cardiac anatomy (e.g., the cardiac surface).

In embodiments of the disclosure, omnipolar techniques can be applied to the unipolar and/or bipolar electrograms that can be measured by a clique of three or more electrodes 17 carried by catheter 13. For instance, each individual electrode 17 within a clique can be used to measure a unipolar electrogram; those of ordinary skill in the art will be familiar with various techniques suitable for defining a representative unipolar electrogram for the clique as a whole (e.g., an average of individual unipolar electrograms). Likewise, each pair of electrodes 17 within a clique can be used to measure a bipolar electrogram. Details of computing E-field loops from these various unipolar and bipolar electrograms, and for generating omnipolar electrograms therefrom, are described in U.S. Pat. Nos. 10,758,137 and 10,194,994; international patent application publication no. WO 2015/130824; and Deno et al., Orientation-Independent Catheter-Based Characterization of Myocardial Activation, IEEE Transactions on Biomedical Engineering, Vol. 64, No. 5, 1067-1077 (May 2017) (“Deno”). Each of the foregoing is hereby incorporated by reference as though fully set forth herein.

As will be apparent from the foregoing description, catheter 13 can be used to simultaneously collect a plurality of electrophysiology data points for the various unipoles and bipoles defined by electrodes 17 thereon. Each such electrophysiology data point includes both localization information (e.g., position of a unipole; position and orientation of a selected bipole or electrode clique) and corresponding electrogram signals (e.g., unipolar, bipolar, and/or omnipolar electrograms).

Catheter 13 (or multiple such catheters) are typically introduced into the heart and/or vasculature of the patient via one or more introducers and using familiar procedures. Indeed, various approaches to introduce catheter 13 into a patient's heart, such as transseptal approaches, will be familiar to those of ordinary skill in the art, and therefore need not be further described herein.

Since each electrode 17 lies within the patient, location data may be collected simultaneously for each electrode 17 by system 8. Similarly, each electrode 17 can be used to gather electrophysiological data from the cardiac surface (e.g., endocardial electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate graphical representations of cardiac geometry (e.g., cardiac surface models) and/or cardiac electrical activity (e.g., electrophysiology maps) from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and the pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., surface electrode pairs 12/14, 16/22, and 18/19) in order to realize catheter navigation in a biological conductor. Alternatively, these orthogonal fields can be decomposed and any pairs of surface electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are exposed to the field from navigational currents and are measured with respect to ground, such as belly patch 21. In practice the catheters within the heart 10 may contain multiple electrodes and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data from the surface electrodes and/or the internal electrodes may be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current signals. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In aspects of the disclosure, system 8 can be a hybrid system that incorporates both impedance-based (e.g., as described above) and magnetic-based localization capabilities. Thus, for example, system 8 can also include a source 30 coupled to one or more magnetic field generators. In the interest of clarity, only two magnetic field generators 32 and 33 are depicted in FIG. 1, but it should be understood that additional magnetic field generators (e.g., a total of six magnetic field generators, defining three generally orthogonal axes analogous to those defined by patch electrodes 12, 14, 16, 18, 19, and 22) can be used without departing from the scope of the present teachings. Likewise, those of ordinary skill in the art will appreciate that, for purposes of localizing catheter 13 within the magnetic fields so generated, can include one or more magnetic localization sensors (e.g., coils).

In some embodiments, system 8 is the EnSite™ X, EnSite™ Velocity™, or EnSite Precision™ electrophysiological mapping and visualization system of Abbott Laboratories (Abbott Park, Illinois). Other localization systems, however, may be used in connection with the present teachings, including for example the RHYTHMIA HDX™ mapping system of Boston Scientific Corporation (Marlborough, Massachusetts), the CARTO navigation and location system of Biosense Webster, Inc. (Irvine, California), the AURORA® system of Northern Digital Inc. (Waterloo, Ontario), Stereotaxis, Inc.'s NIOBE® Magnetic Navigation System (St. Louis, Missouri), the Affera™ Mapping and Ablation System of Medtronic plc (Minneapolis, Minnesota), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Aspects of the disclosure relate to identifying critical isthmus for treatment of tachycardia via application of ablation therapy. System 8 (e.g., computer system 20) can therefore include a hardware- and/or software-based critical isthmus processing unit 58 as further described below.

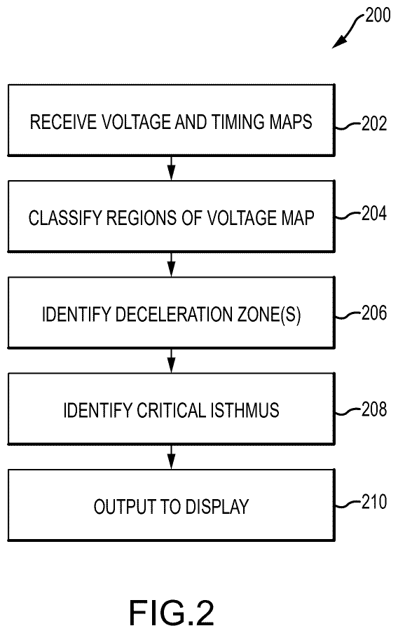

Exemplary methods according to aspects of the instant disclosure will be explained with reference to the flowchart 200 of representative steps presented as FIG. 2. In some embodiments, for example, flowchart 200 may represent several exemplary steps that can be carried out by electroanatomical mapping system 8 of FIG. 1 (e.g., by processor 28 and/or critical isthmus processing unit 58). It should be understood that the representative steps described below can be hardware-implemented (e.g., as an application-specific integrated circuit (ASIC) or field-programmable gate array (FPGA)), software-implemented (e.g., as a series of programming instructions executed on one or more processing units), or implemented in a combination of hardware and software.

As described above, system 8 can localize electrodes 17 and/or multi-electrode catheter 13 using techniques that will be familiar to those of ordinary skill in the art. Thus, electrophysiological data (e.g., unipolar electrograms, bipolar electrograms, and/or omnipolar electrograms) can be associated with the cardiac location at which they were measured and/or computed. The association of electrophysiological data (e.g., electrogram signals) with the cardiac location at which such data were gathered (or computed) is commonly referred to as an “electrophysiology data point” (or “EP data point”). A collection or set of EP data points can be used to define an “electrophysiology map,” which, in turn, can be output to display 23 of system 8.

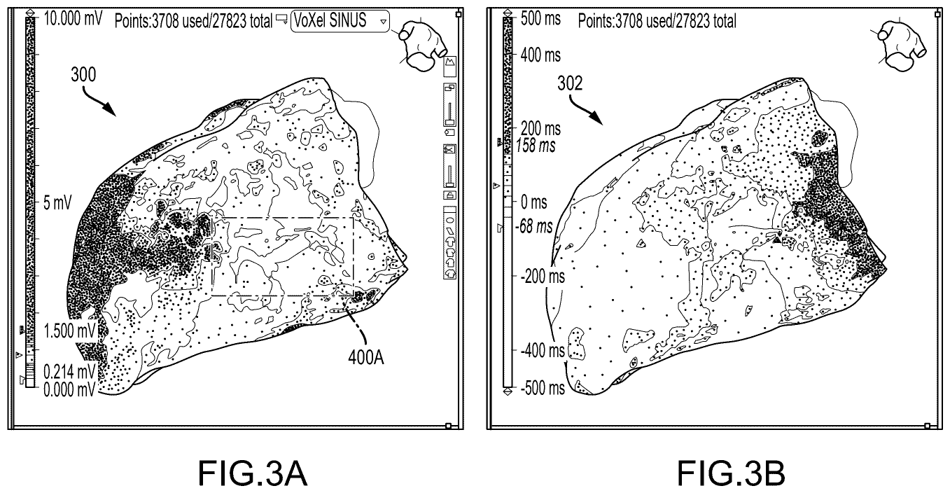

Various electrophysiology maps will be known to those of skill in the art. In embodiments of the instant disclosure, however, system 8 receives a voltage map (e.g., a peak-to-peak voltage map) of a portion of a patient's heart and a timing map (e.g., a local activation timing (LAT) map or an isochronal late activation map (ILAM)) of the portion of the patient's heart in block 202. Insofar as voltage and timing maps will be familiar to those of ordinary skill in the art, there is no need for a detailed discussion thereof in the instant disclosure. To aid in understanding, and for purposes of illustration only, FIG. 3A depicts a representative peak-to-peak voltage map 300 and FIG. 3B depicts a representative ILAM 302.

In block 204, system 8 uses the voltage map received in block 202 to classify the portion of the patient's heart into one or more scar regions, one or more healthy regions, and one or more low-voltage regions. More particularly, system 8 classifies the EP data points of the voltage map as either healthy, scar, or low-voltage; clusters of similarly-classified EP data points (that is, similarly-classified EP data points that are within a preset threshold distance of each other) define corresponding regions in the portion of the patient's heart. The preset threshold distance may be a sphere or circle having a radius of about 1 cm, though it is contemplated that the threshold distance may be adjustable at a practitioner's discretion.

In embodiments of the disclosure, system 8 classifies the EP data points as either healthy, scar, or low-voltage based on how the voltage (e.g., the peak-to-peak voltage) at a given EP data point compares to preset voltage ranges and/or threshold voltage values. For instance, in studies of ventricular tachycardia, system 8 can classify data points having a voltage below about 0.5 mV as scar, data points having a voltage above about 1.5 mV as healthy, and data points having a voltage between about 0.5 mV and about 1.5 mV as low-voltage.

It should be understood, however, that the preset voltage ranges and/or threshold voltage values may differ depending on the tachycardia under study. For instance, in studies of atrial tachycardia, system 8 can classify data points having a voltage below about 0.1 mV as scar, data points having a voltage above about 0.5 mV as healthy, and data points having a voltage between about 0.1 mV and about 0.5 mV as low-voltage.

It is also contemplated that the voltage ranges and/or threshold voltage values may be user-defined or user-adjusted from presets defined within system 8, such as the exemplary ventricular tachycardia and atrial tachycardia ranges and thresholds described above. It is desirable, however, for the voltage ranges and/or threshold voltage values to be preset or selected such that the majority of EP data points are classified as either scar or low-voltage; in some aspects of the disclosure, the voltage ranges and/or threshold voltage values are preset or selected such that about 80% or more of the EP data points are classified as either scar or low-voltage.

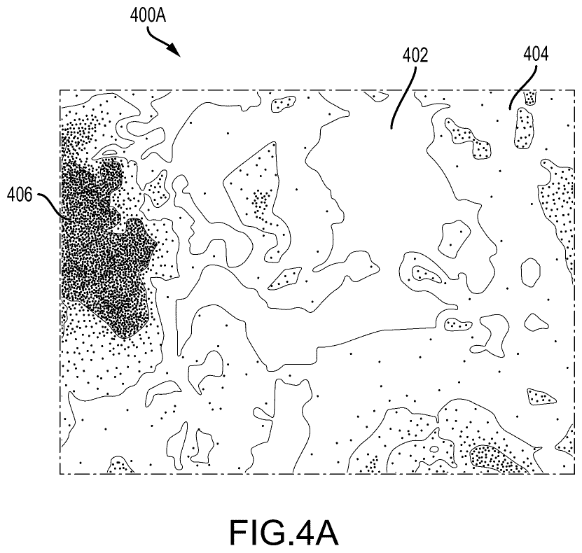

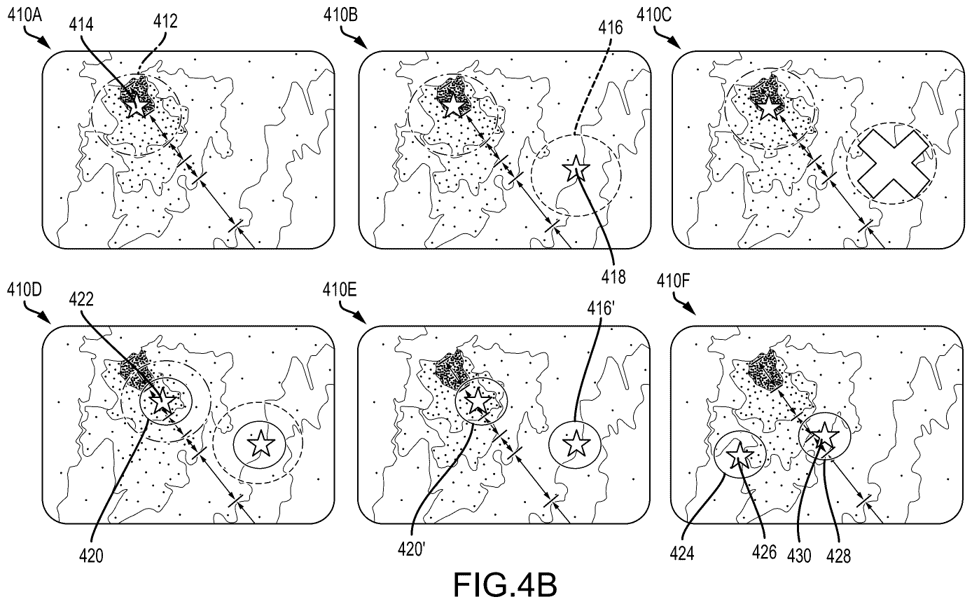

In any event, the output of block 204 is an identification of one or more low-voltage regions within the portion of the patient's heart. FIG. 4A is a close-up of box 400A in FIG. 3A indicating a low-voltage region 402 distributed irregularly within scar tissue 404. A region of healthy tissue 406 is also visible.

In block 206, system 8 uses the timing map received in block 202 to identify one or more deceleration zones in the portion of the patient's heart. Those of skill in the art will appreciate that, in a timing map, such as a LAT map or ILAM, constituent EP data points can be grouped into a preset number (often eight) of isochronal groups. As used herein, the term “deceleration zone” refers to a region of preset size, such as a sphere or circle having a radius of about 1 cm (though this may be user-adjusted), within which the isochronal groups are tightly packed or crowded, thus indicating a rapid reduction in local conduction velocity.

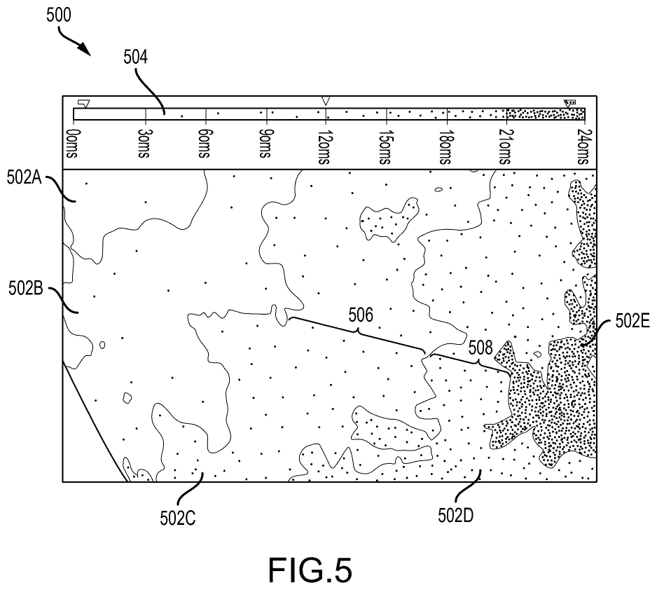

FIG. 5 depicts how deceleration of the cardiac activation wavefront can be identified using isochronal groups in an ILAM 500. Within the portion of ILAM 500 shown in FIG. 5, there are visible several isochronal groups 502A, 502B, 502C, 502D, and 502E. Consider isochronal groups 502C and 502D. As shown in scale 504, isochronal groups 502C and 502D represent the same range of activation timing values. Brackets 506 and 508, however, demonstrate that isochronal group 502D extends over about half the distance of isochronal group 502C on the surface of the patient's heart. Thus, the activation wavefront is moving more slowly in isochronal group 502D than in isochronal group 502C.

While FIG. 5 illustrates how crowding of isochronal groups can be interpreted as deceleration of a cardiac activation wavefront, system 8 can use various criteria to determine whether the isochronal groups are sufficiently tightly packed within a given region to constitute a deceleration zone for purposes of the instant disclosure. For instance, system 8 can identify the region having the greatest number of isochronal groups as a deceleration zone (or regions, if multiple regions have the same number of isochronal groups). As another example, system 8 can identify a region as a deceleration zone if that region contains at least half of the total number of isochronal groups (e.g., four or more). The two criteria can also be combined (e.g., system 8 identifies a region as a deceleration zone if it has the greatest number of isochronal groups and the greatest number of isochronal groups is more than half the total number of isochronal groups).

Identification of deceleration zones in various scenarios according to the foregoing description can be further understood (in two-dimensions for case of illustration) with reference to FIG. 4B. In upper-left frame 410A, a first area (e.g., a sphere or circle having radius of about 1 cm) 412 is defined about a first EP data point 414. First area 412 includes five isochronal groups.

In upper-middle frame 410B, a second area (e.g., a sphere or circle having a radius of about 1 cm) 416 is defined about a second EP data point 418. Second area 416 includes four isochronal groups. Because first area 412 includes more isochronal groups (five) than second area 416 (four), as between the two, first area 412 would be the deceleration zone; the elimination of second area 416 as a deceleration zone is shown in upper-right frame 410C.

In lower-left frame 410D, a third area (e.g., a sphere or circle having a radius of about 1 cm) 420 is defined about a third EP data point 422. Like second area 416, third area 420 includes four isochronal groups. In this scenario, it is contemplated that the size of the area can be reduced (e.g., to a sphere or circle having a radius of about 0.5 cm), defining a revised second area 416′ about second EP data point 418 and a revised third area 420′ about third EP data point 422 (see also lower-middle frame 410E). Revised second area 416′ includes two isochronal groups, while revised third area 420′ still includes four isochronal groups. Thus, as between revised second area 416′ and revised third area 420′, system 8 would identify revised third area 420′ as the deceleration zone.

Lower-right frame 410F illustrates an alternative approach to a scenario where two areas about EP data points include the same number of isochronal groups. In frame 410F, both fourth area 424 about fourth EP data point 426 and fifth area 428 about fifth EP data point 430 include three isochronal groups even after reduction in the size of the area (e.g., from a sphere or circle having a radius of about 1 cm to a sphere or circle having a radius of about 0.5 cm). Thus, system 8 can identify both fourth area 424 and fifth area 428 as deceleration zones (assuming the criterion that requires a deceleration zone to have at least half the total number of isochronal groups is not in use), at least until an area with a greater number of isochronal groups is located.

In any event, the output of block 206 is an identification of one or more deceleration zones within the portion of the patient's heart.

It is understood that low-voltage regions that also exhibit slow conduction (that is, deceleration zones) correlate well to critical isthmus locations, and are therefore desirable targets for the termination of tachycardia via ablation therapy. To this end, in block 208, system 8 uses the outputs of blocks 204 and 206 to identify one or more critical isthmus locations in the portion of the patient's heart. Thus, rather than requiring a practitioner to manually identify critical isthmus locations via visual interpretation of voltage and/or timing maps, system 8 can algorithmically identify critical isthmus locations for the practitioner's consideration via operation of processor 28 and critical isthmus processing unit 58 as described in further detail below.

In certain embodiments, block 208 uses a trained machine learning model to automatically identify critical isthmus locations. This model is referred to herein as a “classifier.” In addition to identifying critical isthmus locations, the classifier can also output a probability that ablation at an identified location will terminate tachycardia. This probability is referred to herein as a “tachycardia termination confidence value.” In some embodiments of the disclosure, the classifier can identify a location as a critical isthmus location when the location has a tachycardia termination confidence value above a preset threshold, such as about 80%, though it is contemplated that this threshold may be varied at a practitioner's option.

The classifier can employ a convolutional neural network algorithm trained with data from a plurality of subjects that received ablation therapy for treatment of tachycardia. That is, a training data set for the classifier can include voltage and timing maps from such subjects with actual ablation locations annotated, as well as corresponding therapeutic outcomes (e.g., whether the tachycardia was successfully terminated via ablation at the annotated locations).

Within the training data set, images of regions around ablation locations (including their associated voltage and timing information) can be normalized and converted to one-hot encoding. The convolutional neural network architecture can be defined with an input layer that matches the image size, followed by multiple convolutional layers featuring rectified linear unit (ReLU) activation and max pooling.

A softmax activation function can be employed in the output layer to accommodate multiple possible critical isthmus locations:

$$\hat{y} = softmax ( o ) , \hat{{y}_{ι}} = \frac{exp ( {o}_{i} )}{{\sum }_{j} exp ( {o}_{j} )} ,$$

where y are the ground-truth labels, ŷ is the predicted output, o=Wx+b is the output layer, x is the normalized matrix for an image of one region in the training data set, W is the weight matrix applied to input feature x learned during training, and b is a bias vector to allow the model to better fit the data.

Cross-entropy loss can be used as the loss function for training the convolutional neural network model because it is suitable for measuring the performance of a model where the output is a probability (e.g., a probability that ablation at a particular location will terminate tachycardia). The loss function can be defined as:

$$l ( y , \hat{y} ) = - {\sum }_{j}^{q} {y}_{j} log {\hat{y}}_{J} .$$

In predicting critical isthmus locations, the sum of the probabilities of all possible critical isthmus locations equals one. Since y is a one-hot vector of length q, the sum over all its coordinates j vanishes for all but one term.

ADAN (a method for stochastic optimization) can be used as the optimization algorithm to train the convolutional neural network model due to its advantages in adaptive learning, computational efficiency, and robustness in fine-tuning.

After each evolution, the convolutional neural network model can be validated using a validation data set. Similar to the training data set, the validation data set can include voltage and timing maps from subjects that received ablation therapy for treatment of tachycardia, with ablation locations annotated, as well as associated therapeutic outcomes (e.g., whether the tachycardia was successfully terminated via ablation at the annotated locations).

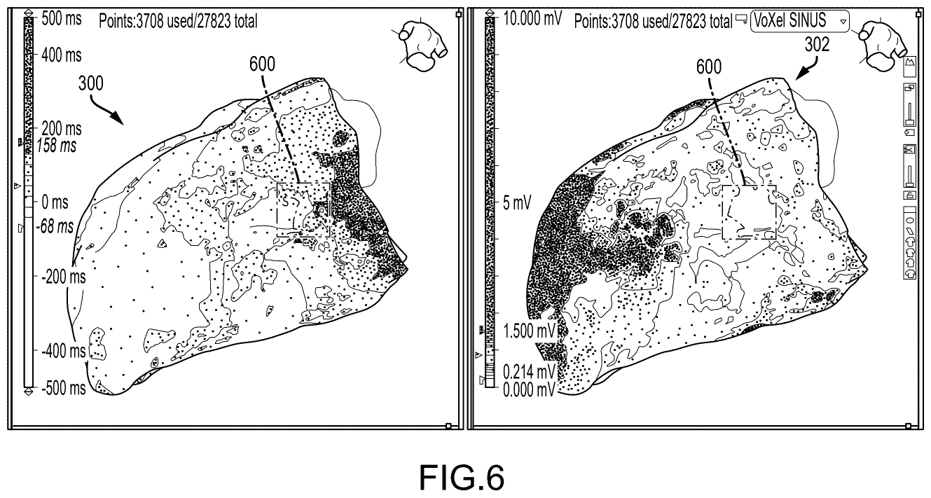

The convolutional neural network model with the best validation accuracy can be selected as the classifier used by system 8 (e.g., processor 28 and/or critical isthmus processing unit 58) in block 208. In turn, the output of block 208—that is, the identification of one or more critical isthmus locations, and optionally their associated tachycardia termination confidence values—may be output to display 23 (e.g., overlaid on a three-dimensional geometric model of the heart) in block 210. For instance, FIG. 6 shows peak-to-peak voltage map 300 of FIG. 3A and ILAM 302 of FIG. 3B with a critical isthmus location 600 identified by the classifier annotated thereon. The ultimate decision of whether to ablate at any such identified location, however, is committed to the practitioner's expertise.

Although several embodiments have been described above with a certain degree of particularity, those skilled in the art could make numerous alterations to the disclosed embodiments without departing from the spirit or scope of this invention.

For example, the teachings herein can be applied in real time (e.g., during an electrophysiology study) or during post-processing (e.g., to electrophysiology signals collected during an electrophysiology study performed at an earlier time).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

Aspects of the disclosure are also set out in the following numbered clauses:
1. A computer-implemented method of identifying critical isthmus for treatment of tachycardia via application of ablation therapy, the method comprising:
   receiving a voltage map of a portion of a patient's heart and a timing map of the portion of the patient's heart at an electroanatomical mapping system;
   identifying, using the electroanatomical mapping system, one or more low-voltage regions in the portion of the patient's heart from the voltage map;
   identifying, using the electroanatomical mapping system, one or more deceleration zones in the portion of the patient's heart from the timing map; and
   generating an output that comprises an identification of one or more critical isthmus locations in the portion of the patient's heart using a trained classifier and based on the identified one or more low-voltage regions in the portion of the patient's heart and the identified one or more deceleration zones in the portion of the patient's heart.

The step of generating an output may be performed using a critical isthmus processing unit. The critical isthmus processing unit may comprise the trained classifier. The trained classifier may be trained with data from a plurality of subjects that received ablation therapy for treatment of tachycardia.
2. The method according to clause 1, wherein the output further comprises a tachycardia termination confidence value for each of the one or more identified critical isthmus locations.
3. The method according to clause 1 or 2, wherein identifying, using the electroanatomical mapping system, one or more low-voltage regions in the portion of the patient's heart from the voltage map comprises the electroanatomical mapping system identifying one or more regions having a peak-to-peak voltage within a preset range as the one or more low-voltage regions.
4. The method according to clause 3, further comprising the electroanatomical mapping system receiving a user input to define the preset range.
5. The method according to clause 3 or 4, wherein the preset range is between 0.5 mV and 1.5 mV.
6. The method according to any of clauses 1 to 5, wherein identifying, using the electroanatomical mapping system, one or more deceleration zones in the portion of the patient's heart from the timing map comprises the electroanatomical mapping system identifying one or more regions of a preset size including a greatest number of isochronal groups as the one or more deceleration zones.
7. The method according to clause 6, wherein the timing map includes a preset number of isochronal groups and wherein each of the one or more deceleration zones includes at least half of the preset number of isochronal groups.
8. The method according to clause 6 or 7, further comprising the electroanatomical mapping system receiving a user input to define the preset size.
9. The method according to clause 6, 7 or 8, wherein the preset size is a 1 cm radius sphere.
10. The method according to any of clauses 1 to 9, wherein the critical isthmus processing unit comprises a convolutional neural network algorithm.
11. An electroanatomical mapping system for identifying critical isthmus for treatment of tachycardia via application of ablation therapy, the electroanatomical mapping system comprising:
a critical isthmus processing unit comprising a classifier trained with data from a plurality of subjects that received ablation therapy for treatment of tachycardia, wherein the critical isthmus processing unit is configured to:
   receive a voltage map of a portion of a patient's heart and a timing map of the portion of the patient's heart;
   identify one or more low-voltage regions in the portion of the patient's heart from the voltage map;
   identify one or more deceleration zones in the portion of the patient's heart from the timing map; and
   generate an output, based on the identified one or more low volage regions in the portion of the patient's heart and the identified one or more deceleration zones in the portion of the patient's heart, using the classifier, wherein the output comprises an identification of one or more critical isthmus locations in the portion of the patient's heart.
12. The system according to clause 11, wherein the output further comprises a tachycardia termination confidence value for each of the one or more identified critical isthmus locations.
13. The system according to clause 11 or 12, wherein each low-volage region of the one or more low-voltage regions has a peak-to-peak voltage within a preset range.
14. The system according to clause 13, wherein the preset range is between 0.5 mV and 1.5 mV.
15. The system according to any of clauses 11 to 14, wherein each deceleration zone of the one or more deceleration zones includes, within a region of the portion of the patient's heart having a preset size, a greatest number of isochronal groups.
16. The system according to clause 15, wherein each deceleration zone of the one or more deceleration zones includes, within the region of the patient's heart having the preset size, at least half of a preset total number of isochronal groups.
17. The system according to clause 15 or 16, wherein the region of the portion of the patient's heart having the preset size comprises a 1 cm radius spherical region of the portion of the patient's heart.
18. The system according to any of clauses 11 to 17, wherein the critical isthmus processing unit comprises a convolutional neural network algorithm.
19. A method of using an electroanatomical mapping system to identify critical isthmus for treatment of tachycardia via application of ablation therapy, the method comprising:
providing the electroanatomical mapping system with a critical isthmus processing unit, wherein the critical isthmus processing unit includes a classifier trained with data from a plurality of subjects that received ablation therapy for treatment of tachycardia, and wherein the critical isthmus processing unit is further programmed to execute a series of steps comprising:
   receiving a voltage map of a portion of a patient's heart and a timing map of the portion of the patient's heart;
   classifying the portion of the patient's heart into one or more scar regions, one or more healthy regions, and one or more low-volage regions using the electroanatomical mapping system using the voltage map of the portion of the patient's heart;
   identifying one or more deceleration zones in the portion of the patient's heart using the timing map of the portion of the patient's heart; and
   generating an output using the classifier, wherein the output comprises an identification of one or more critical isthmus locations in the portion of the patient's heart based on the identified one or more low volage regions in the portion of the patient's heart and the identified one or more deceleration zones in the portion of the patient's heart.
20. The method according to clause 19, wherein the output further comprises a tachycardia termination confidence value for each of the one or more identified critical isthmus locations.

What is claimed is:

1. A computer-implemented method of identifying critical isthmus for treatment of tachycardia via application of ablation therapy, the method comprising:
receiving a voltage map of a portion of a patient's heart and a timing map of the portion of the patient's heart at an electroanatomical mapping system;
identifying, using the electroanatomical mapping system, one or more low-voltage regions in the portion of the patient's heart from the voltage map;
identifying, using the electroanatomical mapping system, one or more deceleration zones in the portion of the patient's heart from the timing map; and
generating an output, based on the identified one or more low-voltage regions in the portion of the patient's heart and the identified one or more deceleration zones in the portion of the patient's heart, using a critical isthmus processing unit comprising a classifier trained with data from a plurality of subjects that received ablation therapy for treatment of tachycardia,
wherein the output comprises an identification of one or more critical isthmus locations in the portion of the patient's heart.

2. The method according to claim 1, wherein the output further comprises a tachycardia termination confidence value for each of the one or more identified critical isthmus locations.

3. The method according to claim 1, wherein identifying, using the electroanatomical mapping system, one or more low-voltage regions in the portion of the patient's heart from the voltage map comprises the electroanatomical mapping system identifying one or more regions having a peak-to-peak voltage within a preset range as the one or more low-voltage regions.

4. The method according to claim 3, further comprising the electroanatomical mapping system receiving a user input to define the preset range.

5. The method according to claim 3, wherein the preset range is between 0.5 mV and 1.5 mV.

6. The method according to claim 1, wherein identifying, using the electroanatomical mapping system, one or more deceleration zones in the portion of the patient's heart from the timing map comprises the electroanatomical mapping system identifying one or more regions of a preset size including a greatest number of isochronal groups as the one or more deceleration zones.

7. The method according to claim 6, wherein the timing map includes a preset number of isochronal groups and wherein each of the one or more deceleration zones includes at least half of the preset number of isochronal groups.

8. The method according to claim 6, further comprising the electroanatomical mapping system receiving a user input to define the preset size.

9. The method according to claim 6, wherein the preset size is a 1 cm radius sphere.

10. The method according to claim 1, wherein the critical isthmus processing unit comprises a convolutional neural network algorithm.

11. An electroanatomical mapping system for identifying critical isthmus for treatment of tachycardia via application of ablation therapy, the electroanatomical mapping system comprising:
a critical isthmus processing unit comprising a classifier trained with data from a plurality of subjects that received ablation therapy for treatment of tachycardia, wherein the critical isthmus processing unit is configured to:
receive a voltage map of a portion of a patient's heart and a timing map of the portion of the patient's heart;
identify one or more low-voltage regions in the portion of the patient's heart from the voltage map;
identify one or more deceleration zones in the portion of the patient's heart from the timing map; and
generate an output, based on the identified one or more low volage regions in the portion of the patient's heart and the identified one or more deceleration zones in the portion of the patient's heart, using the classifier, wherein the output comprises an identification of one or more critical isthmus locations in the portion of the patient's heart.

12. The system according to claim 11, wherein the output further comprises a tachycardia termination confidence value for each of the one or more identified critical isthmus locations.

13. The system according to claim 11, wherein each low-volage region of the one or more low-voltage regions has a peak-to-peak voltage within a preset range.

14. The system according to claim 13, wherein the preset range is between 0.5 mV and 1.5 mV.

15. The system according to claim 11, wherein each deceleration zone of the one or more deceleration zones includes, within a region of the portion of the patient's heart having a preset size, a greatest number of isochronal groups.

16. The system according to claim 15, wherein each deceleration zone of the one or more deceleration zones includes, within the region of the patient's heart having the preset size, at least half of a preset total number of isochronal groups.

17. The system according to claim 15, wherein the region of the portion of the patient's heart having the preset size comprises a 1 cm radius spherical region of the portion of the patient's heart.

18. The system according to claim 11, wherein the critical isthmus processing unit comprises a convolutional neural network algorithm.

19. A method of using an electroanatomical mapping system to identify critical isthmus for treatment of tachycardia via application of ablation therapy, the method comprising:
providing the electroanatomical mapping system with a critical isthmus processing unit, wherein the critical isthmus processing unit includes a classifier trained with data from a plurality of subjects that received ablation therapy for treatment of tachycardia, and wherein the critical isthmus processing unit is further programmed to execute a series of steps comprising:
receiving a voltage map of a portion of a patient's heart and a timing map of the portion of the patient's heart;
classifying the portion of the patient's heart into one or more scar regions, one or more healthy regions, and one or more low-volage regions using the electroanatomical mapping system using the voltage map of the portion of the patient's heart;
identifying one or more deceleration zones in the portion of the patient's heart using the timing map of the portion of the patient's heart; and
generating an output using the classifier, wherein the output comprises an identification of one or more critical isthmus locations in the portion of the patient's heart based on the identified one or more low volage regions in the portion of the patient's heart and the identified one or more deceleration zones in the portion of the patient's heart.

20. The method according to claim 19, wherein the output further comprises a tachycardia termination confidence value for each of the one or more identified critical isthmus locations.
